(19) United States
(12) Patent Application Publication
(10) Pub. No.: US 2003/0050635 A1
(43) Pub. Date: Mar. 13, 2003

(54) Embolization systems and techniques for treating tumors

(76) Inventors: Csaba Truckai, Saratoga, CA (US); John H. Shadduck, Tiburon, CA (US); Tass Adorjan, Kecskemet (HU)

Correspondence Address: Csaba Truckai, 19566 Arden Court, Saratoga, CA, 95070, US

(21) Appl. No.: 10/225,029

(22) Filed: Aug. 21, 2002

Related U.S. Application Data
(60) Provisional application No. 60/314,513, filed on Aug. 22, 2001.

(51) Int. Cl. A61B 18/18

(52) U.S. Cl. 606/41

(57) ABSTRACT

This invention relates to medical systems and least invasive techniques for treatment of hypervascularized tumors, both benign and cancerous, by catheter-based embolization of vessels that feed the tumor, for example in a patient's lung. The least invasive techniques typically can be performed by interventional radiologists for treatment of lung tumors. The invention also can be used to treat hypervascularized tumor tissues in other locations in a patient's body, for example, uterine fibroids and liver tumors.

### CROSS-REFERENCE TO RELATED APPLICATIONS

This application also is related to U.S. Patent Application Serial No. 10/198,041 filed Jul. 20, 2002 titled Systems and Techniques for Lung Volume Reduction.

This application claims benefit of Provisional U.S. Patent Application Ser. No. 60/314,513 filed Aug. 22, 2001 (Docket No. CTX-004) having the same title as this disclosure, which is incorporated herein by reference.

### FIELD OF THE INVENTION

This invention relates to medical systems and least invasive techniques for treatment of hypervascularized tumors, both benign and cancerous, by a catheter-based embolization of vessels that feed the tumor, for example in a patient's lung. The least invasive techniques typically can be performed by interventional radiologists to treat lung tumors. The invention also can be used to treat hypervascularized tumor tissues in other locations in a patient's body, for example, uterine fibroids and liver tumors.

### BACKGROUND OF THE INVENTION

In treating lung cancer patients, the therapeutic options are often limited. In most non-small cell lung cancers (NSCLC), the outcomes are satisfactory only in cases of the most localized cancers. Tumor resection surgery is the major potentially curative option for this disease, while radiation therapy can provide a cure only in a small minority of patients. Newer therapies often combine a localized therapy (resection surgery), a regional therapy (radiation) and a systemic treatment (chemotherapy and immunotherapy). Recently, several seemingly effective systemic therapies have included the use of new pharmacological agents, including paclitaxel (Taxol), docetaxel (Taxotere), topotecan, irinotecan, vinorelbine, and gemcitabine. These agents have been shown to be active in the treatment of advanced NSCLC.

Additional therapeutic options are needed for treating tumors in various forms of lung cancer. New techniques are needed for treating hypervascularized tumors in other locations in a patient's body, such as the uterus and liver.

### SUMMARY OF THE INVENTION

The invention relates to a field that is best defined as embolization therapy, wherein minimally invasive catheters are used to create an occlusion in a targeted artery beyond which the physician wishes to restrict blood flow. The objective of embolization therapy is to shrink or starve tumor tissues, whether benign or malignant, which are supplied by blood flow through the targeted artery.

In an exemplary procedure, the apparatus and method of the invention can be used to treat certain tumors in a patient's lung. In the field of lung cancer, physicians generally define four major bronchogenic carcinoma cell types that account for over 95% of primary cancerous tumors: adenocarcinoma, squamous cell carcinoma, undifferentiated large cell carcinoma, and small cell carcinoma. The different cell types occur singly or in combination.

Adenocarcinoma is the most common type of lung cancer, accounting for about 35% of all cases. The majority of adenocarcinomas occur at the periphery of the lung, and, are often asymptomatic until late in their course. They frequently lie just below the pleura, and cause pleural retraction and thickening on x-ray. Often, adenocarcinomas are discovered on routine chest x-rays or in a primary search for distant metastases. Most adenocarcinomas are between 2 and 5 cm at the time of resection. Adenocarcinomas are often subclassified based upon their degree of differentiation into well-differentiated, moderately-differentiated and poorly-differentiated forms. This subclassification is based upon cytologic features, the presence and amount of solid areas, the level of mitotic activity and the presence and amount of necrosis. A principal type of adenocarcinoma that is well-differentiated is a bronchoalveolar carcinoma. Such a bronchioloalveolar carcinoma can manifest as a single peripheral nodule or mass usually in the upper lung. Most commonly, this nodule is well-circumscribed, and typically arises distal to the terminal bronchioles and spreads along the preexisting alveolar septa without causing significant amounts of lung destruction. This form of tumor has a better prognosis for surgical resection than other form of lung cancer, with as many as 30% to 40% of patients undergoing an attempt at surgical resection.

The apparatus and method of the invention can be used to treat lung tumors, for example bronchoalveolar carcinomas as described above. In treating such a lung tumor with the least invasive instruments and techniques of the invention, a microcatheter is advanced to a targeted location in the bronchial artery. In one embodiment, a catheter has a collapsible working end electrode that is deployed to engage the artery wall (at one or more locations) in the bronchial tree to deliver Rf energy to the vessel wall. The application of energy induces the vessel wall to shrink and occlude about the treated region. The occlusion of the vessel will cause the tertiary bronchus that is diseased to shrink and eventually die. Thereafter, the tissue will be resorbed by the patient's body. In other embodiments, the arteries in the bronchial wall can be damaged and occluded by implantable coils, glues or a hydrogel.

In another preferred method of the invention, a hybrid therapy is provided to occlude a targeted blood vessel section. A microcatheter is advanced to a targeted location in the bronchial artery, and thereafter a pharmacologically active agent is released from the catheter working end to flow downstream into the terminal artery portion. The pharmacological agent can be any agent proven effective in treating cancers (e.g., paclitaxel (Taxol), docetaxel (Taxotere), topotecan, irinotecan, vinorelbine, gemcitabine) or any agent known to be effective in causing endothelial cell damage to cause occlusion of the entire terminal artery portion.

The apparatus and method of the invention also can be used to treat hypervascularized tumors elsewhere in a patient's body—that is, any benign or malignant tumor that is characterized by having a large number of blood vessels feeding it. Such tumors can occur in the liver, uterus, brain and other parts of a human body.

In another method of the invention, the apparatus and method may be used to occlude bronchial arteries for the purpose of reducing lung volume in emphysema patients. Emphysema is a debilitating illness brought about by the destruction of lung tissue that afflicts up to 10% of the population over 50 years old. Emphysema is most commonly caused by cigarette smoking and, in some cases, by a genetic deficiency of the enzyme alpha-1-antitrypsin, a protective antiprotease. The condition is characterized by destruction of the alveoli, which are the microscopic air sacs in the lung where gas exchange takes place. Destruction of these air sacs makes it difficult for the body to obtain oxygen and to get rid of carbon dioxide.

In emphysema, there is a progressive decline in respiratory function due to a loss of lung elastic recoil with a decrease of expiratory flow rates. The damage to the microscopic air sacs of the lung results in air-trapping and hyperinflation of the lungs. As the damaged air sacs enlarge, they push on the diaphragm making it more difficult to breathe. The enlarged air sacs also exert compressive forces on undamaged lung tissues, which further reduces gas exchange by the undamaged lung portions. These changes produce the major symptom emphysema patients suffer—dyspnea (shortness of breath) and difficulty of expiration. Current pharmacological treatments for emphysema include bronchodilators to improve airflow. Also, oxygen therapy is used for patients with chronic hypoxemia.

Recently, a surgical procedure called lung volume reduction surgery (LVRS) has been developed to alleviate symptoms of advanced chronic obstructive lung disease that results from emphysema. The surgery can be either open or endoscopic wherein the most severely emphysematous lung tissue is resected. The development of LVRS was based on the observation that emphysema causes the lung to expand and compress normal functioning lung tissue. If the diseased lung tissue were removed, it was believed that the additional space in the chest cavity would allow the normal lung tissue to expand and carry on gas exchange. LVRS was first introduced in the 1950's but was initially abandoned due to a high operative mortality, primarily due to air leakage. One of the main difficulties of the procedure is suturing the resected lung margin in an airtight manner. Normally there is a vacuum between the ribs and the lungs which helps to make the lungs expand and fill with air when the chest wall expands. If an air leak allows air in the potential space between the ribs and lungs—then the vacuum effect will disappear and the lungs will sag upon chest expansion making it increasingly difficult to inflate the lungs and perform gas exchange.

Surgeons typically remove about 20-30% of each lung in a manner that takes advantage of the heterogeneity of emphysema in which the lesions are usually more severe at the apices and less severe at the lung bases. During the course of surgery, one lung is continually ventilated while the lumen of the contralateral lung is clamped. Subsequently, normal areas of the lung deflate as blood flows past the alveoli and resorbs oxygen, while emphysematous portions of the lung with less blood flow and reduced surface area remain inflated and are targeted for resection. The more recent procedures use bovine pericardium or other biocompatible films to buttress a staple line along the resected lung margin to minimize air leaks.

LVRS improves function of the lung by restoring pulmonary elastic recoil and correcting over-distention of the thorax and depression of the diaphragm. Thus, the objective of LVRS is to provide the patient with improved respiratory mechanics and relief from severe shortness of breath upon exertion. Many patients have reported benefits such as improved airflow, increased functional lung capacity and an improved quality of life. As in any major thoracic procedure, there are many risks, including fever, wound infections, wound hematomas, postoperative fatigue, and tachycardia. The recuperation period following LVRS varies from person to person, but most patients remain in the hospital for two weeks following surgery. The patient then must endure a regime of physical therapy and rehabilitation for several additional months. Further, the duration of the improvement in lung function following resection is not yet completely known-but there is a suggestion that lung function begins to decline two years after LVRS. Despite optimistic reports, the morbidity, mortality and financial costs associated with LVRS appear to be high, with some studies indicating mortality rates ranging from 4-17%.

The invention advantageously provides an apparatus and least invasive method for reduction or elimination of lung tumors by occlusion of arteries that feed the tumor tissue.

The invention provides least invasive means for reduction or elimination of hypervascularized tumors in any location in a patient's body, for example, the lung, uterus, liver or brain.

The invention provides means for permanent occlusion of an extended section of an artery in a patient's body by (i) dispersion of a pharmacologically active agent that causes occlusion in throughout a selected terminal portion of an artery; and (ii) placement of an occlusive element with the vessel lumen just upstream from the selected terminal portion.

The invention provides an endovascular catheter with a working end that carries at least one Rf electrode for shrinking and occluding a targeted site in an artery in a bronchial tree.

The invention provides a remote electrical source method that allows the delivery of electrical energy to catheter working end to occlude blood supply to targeted tertiary bronchial portions.

The invention provides a system with feedback control that modulates power delivery to a catheter working end.

The invention provides a catheter working end and method that utilizes an expandable member with first and second portions of a metallic coating that are adapted to serve as a bi-polar electrode arrangement for occluding a bronchial artery.

The invention provides a method for treating lung tumors that is accomplished by an endovascular catheter.

The invention provides a method for treating lung tumors that can greatly reduce the patient's recuperative period and hospital stay.

The invention provides a method for treating lung tumors that can be repeated over a patient's lifetime.

The invention provides a method for treating lung tumors that will allow greatly reduced costs when compared to open or endoscopic LVR procedures.

### BRIEF DESCRIPTION OF THE DRAWINGS

Other objects and advantages of the present invention will be understood by reference to the following detailed description of the invention when considered in combination with the accompanying Figures, in which like reference numerals are used to identify like components throughout this disclosure.

FIG. 1 shows a schematic view patient's respiratory system and a Type “A” system comprising an elongate endovascular catheter for creating an embolism in a blood vessel for treating a diseased lung portion.

FIG. 2A is an enlarged view of the working end of the catheter of FIG. 1 showing an exemplary electrode arrangement deployable from the catheter sleeve.

FIG. 2B is an alternative catheter working end showing an exemplary electrode arrangement carried by a balloon member.

FIG. 3A is a view of the working end of FIG. 2A being deployed in a targeted site in a patient's tertiary bronchus.

FIG. 3B is an enlarged cut-away view of a targeted artery with the working end of FIG. 2A preparing to occlude the vessel.

FIG. 3C is a view similar to FIG. 3B after sealing and occluding the targeted site.

FIG. 4 is an alternative catheter working end shown in a cut-away view of a targeted vessel that deploys an occlusive coil.

FIG. 5 is an alternative catheter working end shown in a cut-away view of a targeted vessel that deploys a volume of a microporous hydrogel to occlude the vessel.

FIG. 6A is a schematic view patient's respiratory system and a Type “B” system of the invention comprising an member that is adapted for deployment through the patient's bronchus for lung volume reduction.

FIG. 6B is an enlarged view of the working end of the catheter of FIG. 6A showing an exemplary electrode arrangement about a balloon surface for performing a method of the invention to occlude blood vessels in the bronchial wall.

FIGS. 7A-7B depicts a Type “C” system and method of the invention for treating a vascularized tumor wherein the working end of the microcatheter is guided endovascularly to a targeted arterial location wherein (i) FIG. 7A depicts the release a selected pharmacologically active agent to downstream blood flow, and (ii) FIG. 7B depicts the deployment of an occlusion device or composition proximal to the released pharmacologically active agent.

FIG. 8 depicts a Type “C” system of the invention for treating a vascularized tumor that is similar to FIG. 7B except that the method only includes deploying an occlusion device or composition at on or more locations in the artery to terminate blood flow to the tumor.

### DETAILED DESCRIPTION OF THE INVENTION

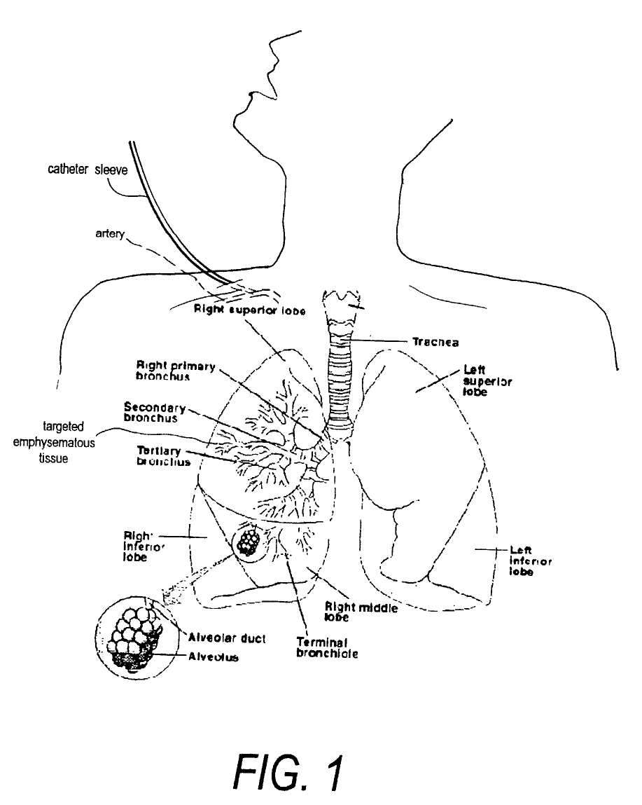

1. Type “A” system for lung volume reduction. FIG. 1 shows a schematic view of a patient's body and lungs 4 with a Type “A” endovascular system 5 introduced from a brachial artery to occlude a targeted site ts in an artery 6 that lies within the wall of the bronchus, which is one of the subdivisions of the trachea that serves to convey air to and from the lungs. The “bronchial tree” as shown in FIG. 1 consists of the primary (right and left) bronchus that branches into the secondary bronchus and tertiary bronchus. In this disclosure, the term tertiary bronchus portions defines any bronchial portions distal to the tertiary branches and leading to terminal bronchioles that may be targeted for reduction.

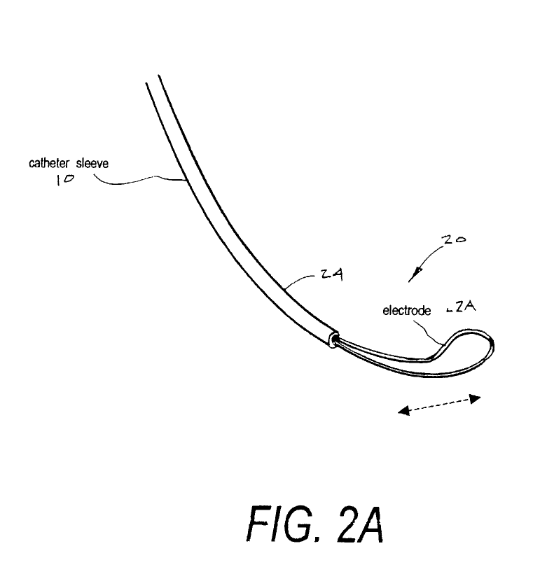

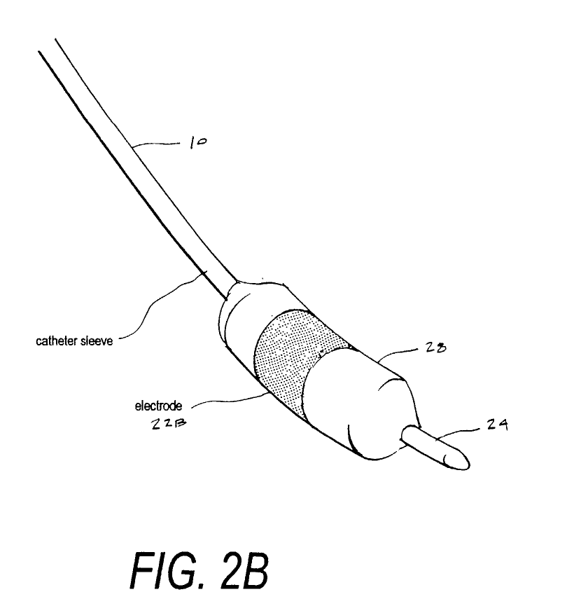

The catheter system has a proximal handle or manifold 9 as is known in the art that is coupled to an elongate microcatheter sleeve 10. FIGS. 2A-2B illustrate enlarged views of exemplary working ends 15 of catheter sleeve 10 that carry an electrode arrangement 20 suitable for engaging the wall of the artery at a targeted site ts. The catheter sleeve 10 can be any suitable diameter, for example, from about 1 Fr. to 6 Fr. In the embodiment of FIG. 2A, the electrode 22A comprises a wire element that is extendable from the distal end 24 of sleeve 10 to assume a slightly expanded cross-sectional dimension compared to the catheter diameter. The electrode wire element 22A is of a shape memory material such as nitinol and can form a loop. The proximal end (not shown) of the electrode wire element 22A is coupled to a remote radio-frequency source 25 as is commonly used in electrosurgical applications. In use, the electrode 22A cooperates with a return electrode, such as a ground pad, coupled elsewhere to the patient's body. FIG. 2B illustrates an alternative embodiment of working end carrying electrode 22B in a band about an expandable member 28 such as an inflatable balloon. The purpose of the working end is simply to provide means for substantial engagement of the electrode arrangement with the vessel wall at the targeted site. Therefore, any single electrode or plurality of electrodes (mono-polar or bi-polar) that can be exposed at the working end of the microcatheter 10 falls within the scope of the invention for performing the method described below.

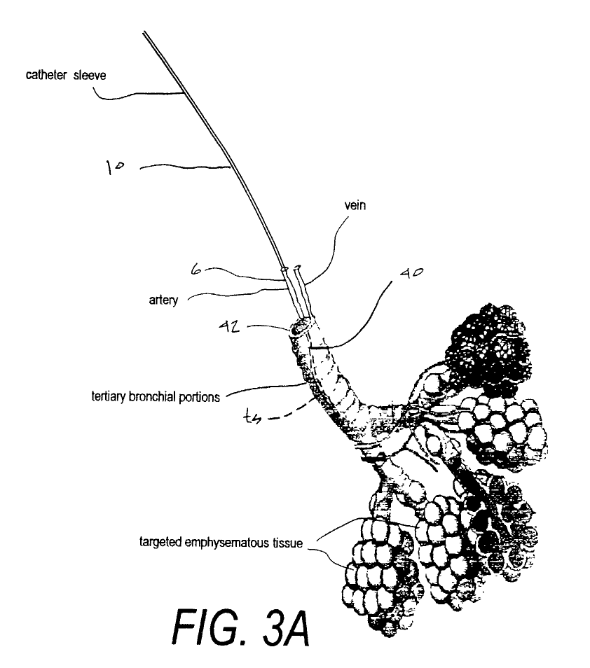

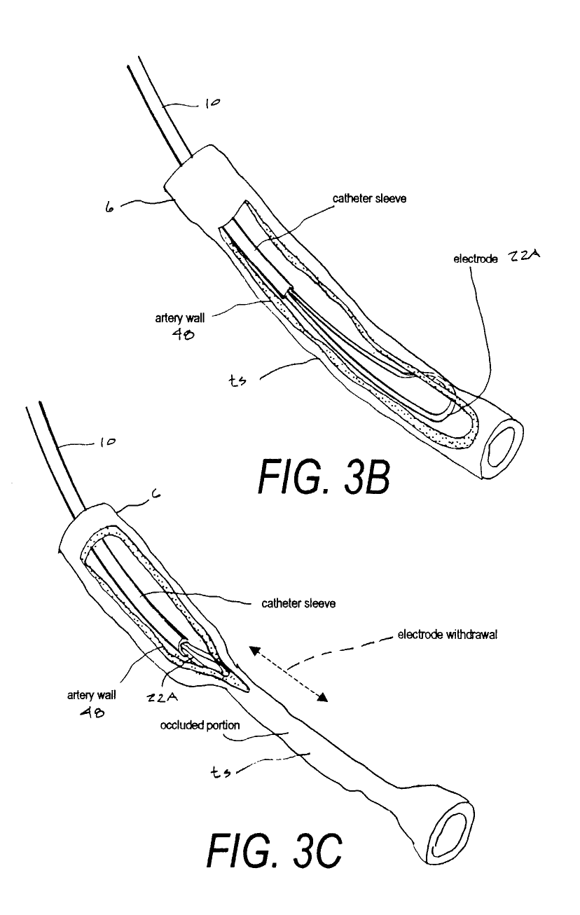

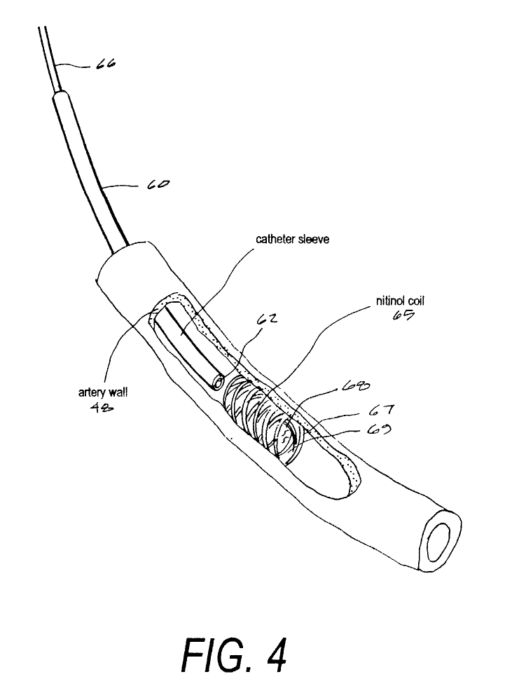

FIGS. 1 & 3A-3C illustrate an exemplary method of utilizing Rf energy to occlude a targeted arterial site ts with the working end 12 of FIG. 2A to accomplish lung volume reduction. FIG. 1 shows the catheter 10 introduced into a brachial artery but any access site is possible. The catheter working end is directed to the targeted site by any imaging means known in the art of endovascular interventions (e.g., ultrasound). FIG. 3A provides a view of the bronchus 40 having a wall 42 that carries an artery 6 in which the targeted site is indicted at ts. The downstream alveoli 44 comprise emphysematous lung tissue which is to be reduced by the method of the invention. FIGS. 3B-3C next shows a cut-away view of the artery 6 alone and the deployment and activation of the Rf electrode loop 22A. In FIG. 3B, the collapsible electrode is extended and thereby presses outwardly against the vessel walls 48. The delivery of Rf energy to the electrode causes thermal effects in the vessel wall thereby inducing the artery to shrink and occlude. FIG. 3C shows the electrode loop 22A being (optionally) withdrawn proximally into the sleeve 10 to provide an elongate seal and occlusion of the artery. This method can be repeated at a number of locations to thereby deprive lung tissue downstream from the targeted sites of blood flow. It is believed that the downstream emphysematous tissue will then wither and slowly be resorbed by the body thus resulting in an effective reduction in lung volume by shrinking and resorption of such damaged tissue. Thus, one method of the invention includes any occlusion of targeted sites in arteries that supply tertiary bronchus portions by application of energy thereto from the working end of a microcatheter. It can be appreciated that the expansion member of FIG. 2B and its electrode 22B can be similarly utilized to occlude and seal an artery (not shown).

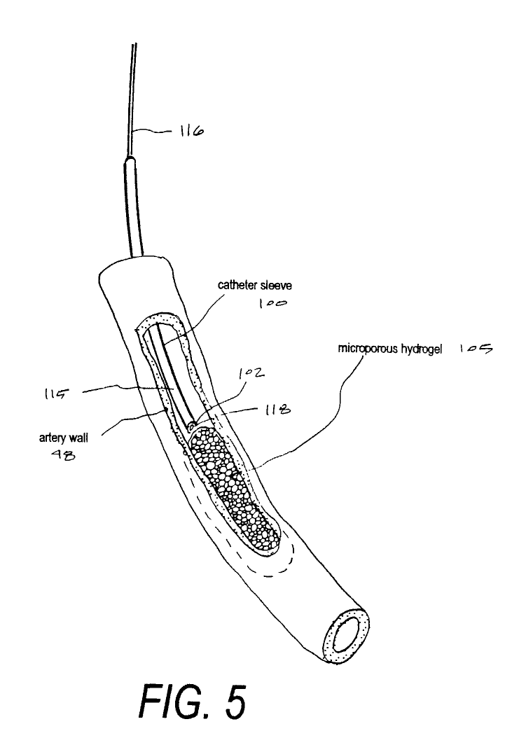

FIG. 4 shows another embodiment of microcatheter sleeve 60 that has a lumen 62 in its working end that carries an occlusion coil 65 that is deployable by a pusher member or mechanism 66. The coil can be of nitinol that is adapted to expand in cross-sectional dimension to engage the vessel wall while at the same time carrying a core of nitinol strands or a polymer film to substantially or completely block blood flow therethrough. Following deployment of the coil 65 at a targeted site, the downstream emphysematous tissue will die and be resorbed by the body to reduce lung volume.

Another embodiment of microcatheter sleeve 80 (not shown) can carry an internal lumen 82 that carries a cyanoacrylate or other similar glue-type biocompatible agent that can be introduced into a patient's blood vessel to occlude the vessel at a targeted site. As described previously, the occlusion can deprive downstream damaged tissue of nourishment causing the dying tissue to be resorbed by the body to reduce lung volume.

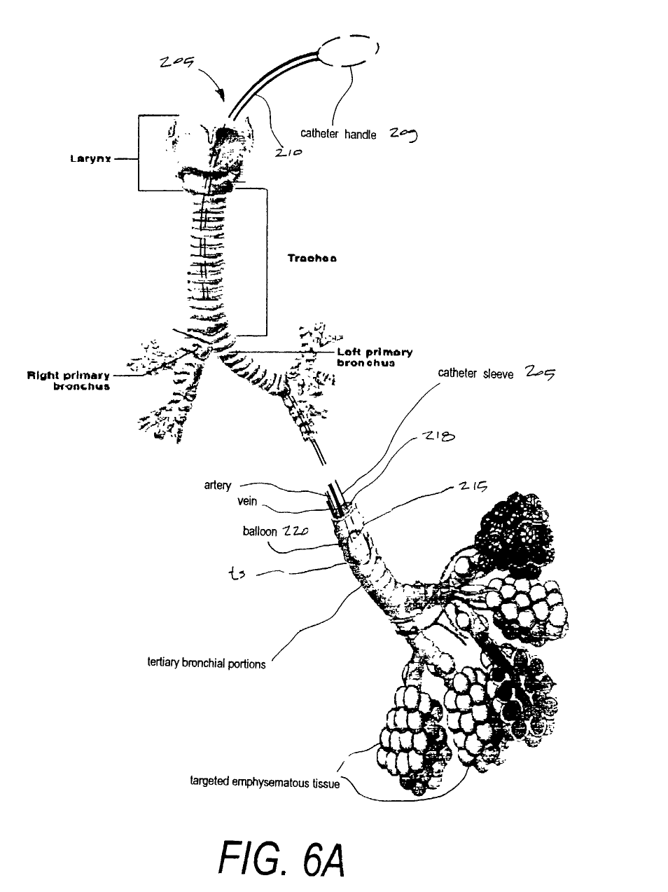

FIG. 5 shows another embodiment of microcatheter sleeve 100 that has an internal lumen 102 that carries a desiccated hydrogel volume 105 that can be deployed into a targeted site in the blood vessel. A microporous or superporous hydrogel is an open cell foam that can be desiccated and collapsed into a thin film or folded into a compressed body for carrying in the lumen 102 of the catheter. Preferably, the hydrogel is resorbable. The hydrogel body 105 can be deployed from the working end 115 of catheter by a pusher member 116 that is actuatable from the catheter handle. A fluid-tight film or gel indicated at 118 is carried about the distal end of lumen 102 to substantially prevent fluids from interacting with the hydrogel before its deployment. After deployment from the catheter, exposure of the hydrogel to a fluid such as blood will expand the hydrogel to a controlled dimension to engage the walls of the artery. A suitable hydrogel can be any biocompatible fast-response gel, for example of PVME, HPC or the like (see, e.g., S. H. Gehrke, Synthesis, Swelling, Permeability and Applications of Responsive Gels in Responsive Gels, K. Du\check{s}ek (Ed.) Springer-Verlag (1993) pp. 86-143).

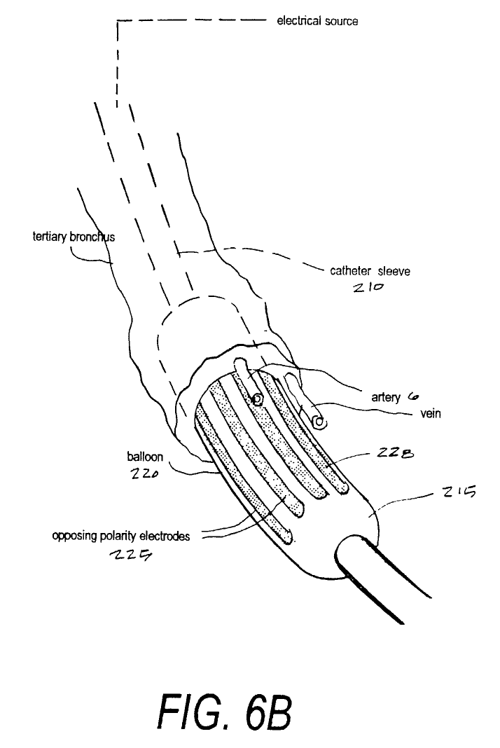

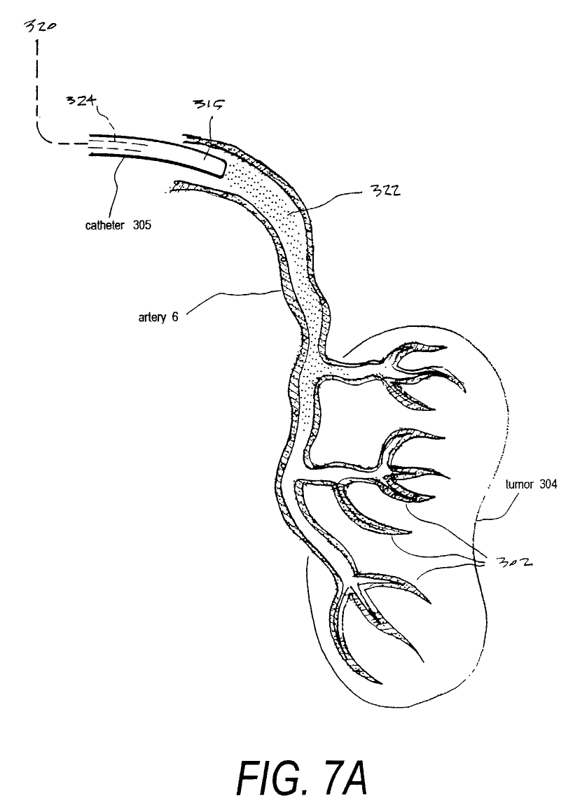

2. Type “B” system for lung volume reduction. FIG. 6A shows another schematic view of a patient's primary and tertiary bronchus with a Type “B” system 200 for LVR that does not comprise an endovascular system—but rather an elongate catheter-type member 205 that is introduced through the patient's bronchial tree to a plurality of targeted sites in tertiary bronchus portions. The objective of the system again targets the artery or arteries within the bronchial wall—but this time from a working end 215 positioned within the lumen 218 of a branch of the bronchus.

In one embodiment (FIG. 6A), the elongate member 205 has a proximal handle 209 coupled to an elongate microcatheter sleeve 210. FIG. 6B illustrates an enlarged view of the exemplary working end 215 of sleeve 210 that carries an expandable balloon member indicated at 220. The surface of the balloon 220 carries a plurality of spaced-apart opposing polarity electrodes 225 (collectively). FIG. 6B shows that the balloon can have exposed electrodes of a thin-layer conductive coating 228. The coating 228 can be any suitable biocompatible material that can be deposited on the balloon wall, such as gold, platinum, silver, palladium, tin, titanium, tantalum, copper or combinations or alloys of such metals, or varied layers of such materials. A preferred manner of depositing a metallic coating on the polymer element comprises an electroless plating process known in the art, such as provided by Micro Plating, Inc., 8110 Hawthorne Dr., Erie, Pa. 16509-4654. The thickness of the metallic coating ranges between about 0.0001 ″to 0.005″.

In a method of use, the working end is advanced to the targeted site. An electrical source is actuated to deliver Rf energy to the electrode arrangement to damage and occlude the artery in the wall of the engaged bronchus portion. The system can further provide at least one feedback control mechanism within a controller for modulating energy delivery to the electrodes. For example, at least one thermocouple can be provided at a surface of the electrode or balloon to measure the temperature of the electrode which is substantially the same as the surface temperature of bronchus wall in contact therewith. The thermocouple is linked to the controller by an electrical lead (not shown). The controller is provided with software and algorithms that are adapted to modulate power delivery from the electrical source to maintain the temperature of the electrodes at a particular level or within a particular temperature range, in response to feedback from the sensor. In a preferred mode of operation, the thermocouple together with feedback circuitry to the controller are used to modulate power delivery to the electrode to maintain a pre-selected temperature level for a selected period of time. The method of invention maintains the surface temperature within a range of about 60° C. to 100° C. More preferably, the surface temperature of the embolic element is maintained within a range of about 80° C. to 100° C. damage and occlude the blood vessels in the wall.

An alternative embodiment of Type “B” system (not shown) for lung volume reduction can also comprise an catheter member that has a working end that is localizable in the patient's tertiary bronchus with means for accessing the artery in the bronchial wall from the airway lumen. Typically, a needle that is extendable from the catheter working end would be utilized—deployable under intra-operative imaging and guidance (e.g., ultrasound). In this embodiment, the working end then could utilize any of the types of systems described in the Type “A” embodiment to occlude the artery: (i) an electrode arrangement coupled to a remote energy source, (ii) a deployable coil, (iii) an injectable cyanoacrylate, or (iv) a deployable volume of a selected hydrogel.

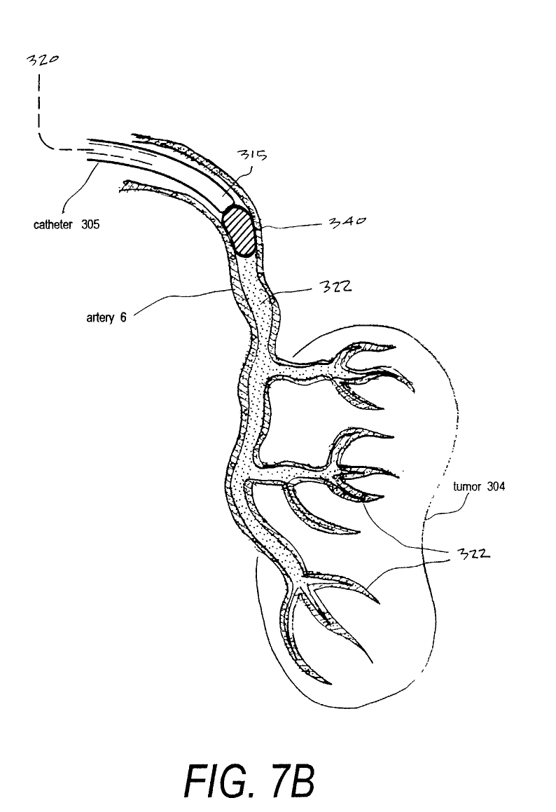

3. Type “C” systems and methods for treatment of vascularized tumors. FIGS. 7A-7B show a Type “C” system 300 that is deployed in a bronchial artery of 6 a patient, for example in a location similar to that of FIG. 3A. In the schematic view of FIG. 7A, the bronchial artery 6 has several branches 302 that feed tumor tissue 304 that resides in a portion of the tertiary bronchus. The method of the invention thus differs from the Types “A” and “B” systems and techniques described above. In FIG. 7A, for example, the tumor 304 may be a type of adenocarcinoma such as a well-differentiated bronchoalveolar carcinoma that manifests as a single peripheral nodule or mass.

FIG. 7A shows elongate catheter member 305 with working end 315 that is positioned within arterial lumen 316. A remote source 320 of a pharmacologically active agent 322 is coupled to a lumen 324 of the catheter 305 to allow delivery of the agent 322 through the working end 315. The source can be a syringe or other pressure source that is adapted for release of the agent 322 into blood flow that can be actuated from the handle of the catheter. FIG. 7A depicts the first step of the method of the invention wherein the released agent 322 flows downstream to the terminal portion of the branched artery that feeds the tumor. FIG. 7B depicts the second step of the method wherein an occlusion member or composition indicated at 340 is deployed from the catheter working end 315 to occlude the artery and capture the pharmacologically active agent 322 in the terminal portion of the artery. The agent 322 can be any of the agents that inhibit the growth of tumor tissue (e.g., paclitaxel (Taxol), docetaxel (Taxotere), topotecan, irinotecan, vinorelbine, gemcitabine) or other that can cause endothelial damage to occlude the entire terminal artery portion. The methods depicted in FIGS. 7A-7B can be repeated in a number of locations. Following such treatment, the tumor tissue will be starved of blood flow and shrink and potentially die. In this method, the occlusion member or composition can be any of those described above, for example, a biocompatible glue, a hydrogel, or any type of embolic coil that can fully occlude the artery such as a self-expanding shape memory nickel titanium alloy stent-type member or any other shape memory material. In addition, the artery can be occluded with an Rf electrode as described previously.

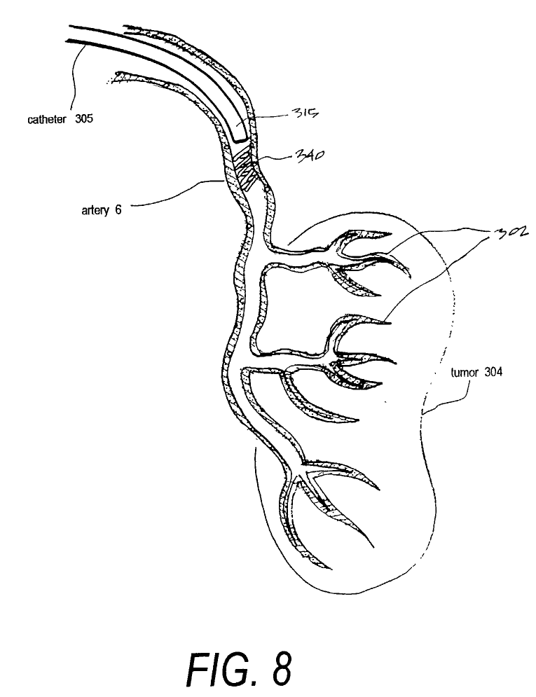

FIG. 8 depicts a method of treating a hypervascularized tumor that is similar to the method depicted in FIGS. 7A-7B, except that the method does not rely on the release of a pharmacological agent and instead only deploys an occlusion member or composition 340 to occlude the artery. While the invention has been described generally with reference to lung tumors, the system and method can be used to treat any vascularized tumors.

Those skilled in the art will appreciate that the exemplary embodiments and descriptions of the invention herein are merely illustrative of the invention as a whole. Specific features of the invention may be shown in some figures and not in others, and this is for convenience only and any feature may be combined with another in accordance with the invention. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention. The appended claims are intended to cover and embrace any and all such modifications, with the limits only being the true purview, spirit and scope of the invention.

What is claimed is:

1. A method for treating vascularized tumor tissue in a patient's body, comprising the steps of:
(a) introducing a catheter working end endovascularly to at least one targeted site in an artery having a terminal artery portion that feeds tumor tissue;
(b) releasing a selected pharmacologically active agent from the catheter working end; and
(c) deploying an occlusive composition from the catheter working end to occlude the artery at the targeted site thereby capturing the selected agent in the terminal artery portion and preventing blood flow within the terminal artery portion.

2. The method of claim 1 wherein step (c) deploys an occlusive composition comprising a self-expanding body of a shape memory material.

3. The method of claim 1 wherein step (c) deploys a self-expanding body of a nickel titanium alloy.

4. The method of claim 1 wherein step (c) deploys an occlusive composition comprising a cyanoacrylate material.

5. The method of claim 1 wherein step (c) deploys an occlusive composition comprising a hydrogel.

6. The method of claim 5 wherein the hydrogel is bioabsorbable.

7. The method of claim 5 wherein the hydrogel is a microporous or superporous gel.

8. The method of claim 1 wherein the selected pharmacologically active agent causes occlusion along the length of the terminal artery portion.

9. The method of claim 1 wherein the selected pharmacologically active agent is selected from the class consisting of paclitaxel, docetaxel, topotecan, irinotecan, vinorelbine, and gemcitabine.

10. A method for treating vascularized tumor tissue in a patient's lung, comprising the steps of:
(a) introducing a catheter working end endovascularly to at least one targeted site in a bronchial artery;
(b) applying energy to said targeted site by delivering electrical current flow at a selected power level to at least one electrode carried at said working end, wherein the application of energy damages and occludes the artery at said targeted site; and
(c) withdrawing the catheter working end from the patient's vasculature wherein the occluded artery causes reduction of blood flow to said vascularized tumor tissue.

11. The method of claim 10 wherein current flow in step (b) is carried to said at least one electrode functioning with a single polarity.

12. The method of claim 10 wherein current flow in step (b) is between first and second spaced apart electrodes functioning with opposing polarities.

13. The method of claim 10 wherein said at least one electrode has a collapsed position and an expanded position and step (a) includes the step of moving the electrode to the expanded position to engage the arterial wall.

14. The method of claim 10 wherein step (b) includes the step of translating said at least one electrode along the arterial wall while delivering electrical current flow thereto.

15. A method for treating vascularized tumor tissue in a patient's body, comprising the steps of:
(a) introducing a catheter working end endovascularly to at least one targeted site in an artery having a terminal artery portion that feeds tumor tissue;
(b) releasing a selected pharmacologically active agent from the catheter working end; and
(c) applying energy to said targeted site by delivering electrical current flow at a selected power level to at least one electrode carried at said working end, wherein the application of energy occludes the artery at said targeted site thereby capturing said selected agent in said terminal artery portion and preventing blood flow within said terminal artery portion.

16. A method for treating vascularized tumor tissue in a patient's body, comprising the steps of:
(a) introducing a catheter working end endovascularly to at least one targeted site in a in artery that feeds tumor tissue;
(b) applying energy to said targeted site by delivering electrical current flow at a selected power level to at least one electrode carried at said working end, wherein the application of energy damages and occludes the artery at said targeted site; and
(c) withdrawing the catheter working end from the patient's vasculature wherein the occluded artery causes reduction of blood flow to said vascularized tumor tissue.
